(19) United States
(12) Patent Application Publication
(10) Pub. No.: US 2022/0240520 A1
(43) Pub. Date: Aug. 4, 2022

(54) Neutralizing Agent with Disinfectant and Deodorizing Effect

(71) Applicant: Ruslan Stanislavovich Shostak, Dnipro (UA)

(72) Inventor: Ruslan Stanislavovich Shostak, Dnipro (UA)

(21) Appl. No.: 17/315,382

(22) Filed: May 10, 2021

(30) Foreign Application Priority Data
Feb. 4, 2021 (UA) A202100432

(51) Int. Cl. A01N 63/50; C12N 9/80; C11D 17/00; C11D 11/00

(52) U.S. Cl. CPC A01N 63/50; C12N 9/80; C12Y 305/01005; C11D 11/0023; C11D 17/0073

(57) ABSTRACT

A composition in the form of a tablet for neutralizing toilet odor and preventing bacterial growth comprising proteolytic enzymes (protease) and a complex of sodium bicarbonate, citric acid, and a surfactant for foaming, as well as EDTA disodium (Trilon B), sodium percarbonate, dye, and fragrance.

### RELATED APPLICATIONS

This application claims priority to Ukrainian Patent Application No. UA a202100432, filed Feb. 4, 2021, which is incorporated herein by reference in its entirety.

### FIELD OF THE INVENTION

The disclosure pertains to deodorants and disinfectants generally and to compositions for neutralizing (eliminating) unpleasant odor from the toilet used without flushing with water.

### BACKGROUND

The problem of unpleasant odors in facilities where people are inside, such as work rooms or private houses, is constantly occurring.

This is especially true of sanitary facilities, bathrooms, and toilets, which are a common element of houses, hotels, long-distance vehicles, restaurants, entertainment centers, theaters, cinemas, etc. The social, professional, and educational premises, where multiple people are regularly present: offices, enterprises of various profiles, schools, medical institutions, etc., that are normally equipped with toilets. The smell of the toilet is unpleasant for users, its presence affects the overall impression of institutions and private housing.

The easiest way to avoid the smell of urine in the toilet is frequent flushing of water in the toilet. However, there is a well-known problem of lack of fresh water, which is increasingly affecting humanity on a global scale. In addition, large volumes of wastewater, especially from the drainage system from sanitary facilities of enterprises of various profiles and of residential housing, despite the high cost of treatment, have a negative impact on the environment. It should also be noted that from time to time there are emergencies in the water supply system and repair works on water mains and houses, which involve turning off the water for a period of several hours to several days, which makes it impossible to flush the toilet during this period. In some cases, noise from the flush system can interfere with residents during meetings, distance learning or recreation.

So, there is a need of overcoming the unpleasant odor in the bathroom with minimal water consumption for drainage.

It should be noted that the unpleasant odor in the toilet is mainly due to the presence in the toilet of urine, especially one component of urine—urea.

Multiple technical solutions aimed at finding ways and compositions for processing (transformation) of sewage in the toilet with minimal water consumption are known.

WO2002095148 discloses biodegradable germicidal and water-saving formulation for toilets, represented by a composition of powder or granules packaged in sachets disintegrating on contact with water and containing benzalkonium chloride, tripolyphosphate sodium, and silicon dioxide. This bag is placed in the toilet, where it acts as a bactericide, suppressing odors, without the need to rinse the toilet with water.

A disadvantage of this solution is that benzalkonium chloride can cause allergic reactions and contact dermatitis. In addition, the formulation of the composition is aimed at destroying bacteria in wastewater but does not eliminate the direct cause of the unpleasant odor—urea, which causes the unpleasant odor. The powder form of the composition is also a disadvantage. The sachet, which contains a measured amount of the composition, can be damaged during transportation and storage. Accordingly, its contents may spill out and the user would inhale the chemicals that are part of the composition.

Another patent application JP2004203818, describes an antimicrobial deodorizing agent for a water-saving toilet and a method of antimicrobial deodorizing treatment. Intended for water-saving toilet an antibacterial deodorant consists of an aqueous solution containing a biguanide antibacterial component and a surfactant. The antibacterial component consists of polyhexamethylenebiguanide hydrochloride in an amount of from 2% to 10% by weight or more, and the surfactant includes one or more epitaxial, nonionic, or amphoteric surfactants as main components up to 50% by weight of antibacterial and deodorant compositions for water-saving toilets, in which the weight ratio of antibacterial component and surfactant is in the range from 1:5 to 5:1. The compositing may also contain a plant extract in an amount of 1% to 40% by weight based on the total weight of the deodorizing agent.

One disadvantage of the above solution is the use of polyhexamethylenebiguanide hydrochloride as an antibacterial component. When this component is inhaled constantly it causes necrotic bronchial lesions, leading to inflammation and pulmonary fibrosis in both experimental animals and humans due to a special sensitivity of bronchi at concentrations even less than 2 ppm (2 mg/l ). In addition, this antimicrobial deodorant is presented in the form of a solution that is not convenient and is intended for use in specially designed water-saving toilets, which limits its use.

The known compositions for eliminating odors from the toilet are made in the form of powders or solutions, which have the disadvantages of inconvenience and use limitations.

The present disclosure provides a bactericidal deodorizing agent in a compact convenient solid form of small size, namely in the form of tablets, to eliminate odor in the toilet.

The tablets for water purification are widely used.

CN106032293 discloses portable tablets for purification of drinking water in the field. As indicated in that document, the subject matter of the patent is a tablet for coagulation, a tablet for disinfection, and a tablet for chlorine removal, wherein the tablet for coagulation contains 78-83% polyaluminum chloride, 10-15% sodium bicarbonate, and 7-10% succinic acid; the disinfectant tablet contains 15-20% sodium dichloroisocyanurate, 42-45% sodium bicarbonate, 25-28% succinic acid, and 8-12% boric acid; the chlorine removal tablet consists of 70-78% sodium sulfite, 2-5% polyvinylpyrrolidone, 12-15% sodium bicarbonate, and 5-10% succinic acid. The contaminated water is made drinkable in the field by coagulation, disinfection, and removal of chlorine.

The disadvantage of this solution is that these tablets are in no way associated with overcoming the unpleasant odor in the bathroom with minimal water consumption for draining.

WO1996032194, discloses the composition in a standard dosage form for periodic purification of a small predetermined volume of contaminated drinking water. The composition includes: (a) an organochlorine compound that serves as a disinfectant and sanitary agent; (b) a solid inorganic metal salt having a trivalent cation and serving as a coagulant-precipitating agent; (c) a solid alkali which is readily soluble in water and very effervescent in the presence of an acid which is used as a dispersing buffer agent; (d) a solid anionic polymer hydrophilic primary colloidal flocculant; (e) a solid cationic polymeric hydrophilic secondary colloidal flocculant; (f) long-fiber cellulose serving as an agglomeration matrix and a pre-filter for absorbing flakes and trapping visible debris and cysts; and (g) a bulk ion exchange absorbent.

The disclosed product is not designed to be used for disinfection and destruction of odors in the toilet.

WO2007107434, 27 Sep. 2007 discloses a composition of tablets for purifying contaminated water to make it drinkable. In particular, the invention relates to highly stable tableted compositions for water purification, which decompose easily and quickly when dispersed in water. The tableted composition for water purification consists of a solid disinfectant containing chlorine and swelling clay.

This composition is also not designed to be used to eliminate odors in the toilet.

The closest analogue of the claimed invention is a solution disclosed in CN106614747. The application discloses a disinfectant tablet containing a protease for the toilet. The disinfectant tablet also consists of from 20 to 30 percent chlorine dioxide, 1 to 3 percent coating passivators, 15 to 20 percent desiccants, 30 to 40 percent foaming agents, 25 to 40 percent acidifiers, 1 up to 5 percent of sustained release substances, from 1 to 5 percent of surfactants and from 1 to 3 percent of biologically active enzymes. The developers of the disinfectant tablet, which contains a toilet protease, believe that it has the following advantages: it is very convenient to use; active substances can be released stably and quickly; the tablet has a strong ability to decompose harmful substances such as bacteria, viruses, chemical toxicants and residual pesticides, thus achieving the efficiency of sterilization and air purification; no pollution; favorable protection of the ecological environment; high stability; environment protection; no toxicity; easy to use. The disinfectant tablet can be widely used in all fields such as medicine, industry, agriculture, and workplaces.

Although this technical solution is the close to the present disclosure, it has a number of significant disadvantages. This tablet has a rather complex formulation and composition, which makes the manufacturing time-consuming. Considering the concentration of chlorine dioxide, the frequent use of this tablet will not be completely safe for consumers, as noted by the inventors. Further, the enzyme used is referred to as a protease, but proteases belong to at least six groups that differ in the structure of the active site of the enzyme: serine, threonine, cysteine, asparagine, metalloproteases, and glutamine, respectively, each having specific biological activity. Therefore, if the inventors use all groups of enzymes, it is not rational in terms of their specific action, and if they use specific enzymes, it is not clear what type of protease was used and what compounds it affects. In addition, the tablet has no deodorizing effect.

### BRIEF SUMMARY

The problem to which the present disclosure is directed is providing compositions for neutralizing odor from the waterless toilet due to the agent with a deodorizing and disinfecting effect, made in the form of a tablet.

The result achieved in the implementation of the technical solution is the destruction and removal of odor from the toilet (or the lavatory pan) due to the presence of urea using a tablet with deodorizing and disinfectant effect without flushing the water during some time.

The present invention is based on data from human physiology and biochemistry. Namely, human urine is a type of excrement, a product of human life, excreted by the kidneys; 91-96% of it consists of water and it also contains nitrogenous breakdown products of proteins: urea, uric and hippuric acids, creatinine, xanthine, urobilin, indican, as well as salts, in particular chlorides, sulfates, and phosphates. Most of urine components are present in it in small amounts, but the urea content for a healthy person is about 10 times higher. Accordingly, the presence of urea in the urine excreted from the body causes an unpleasant odor from the lavatory pan and the toilet in general.

Based on the above facts and user needs, the inventors have proposed a composition with a deodorizing and disinfecting effect that dissolves quickly to eliminate odors from the toilet.

The main active ingredient of the claimed composition is the enzyme protease, which catalyzes the breakdown of urea and, thus, eliminates the cause of odor in the toilet.

Additionally, the composition contains a complex of substances: sodium bicarbonate, citric acid, surfactant, which promote the dissolution and distribution throughout the volume of the solution (in the toilet) of the main active substance—protease—due to the foaming effect.

To soften the water, EDTA disodium (Trilon B) was added to the product. It allows the tablet to dissolve quickly in the liquid, and the composition is evenly distributed throughout the liquid to be treated and reliably neutralizes the urea, thus eliminating the unpleasant odor.

The proposed composition includes sodium percarbonate as a disinfectant, bactericidal, and decontaminating component of the composition.

Additionally, the composition contains a dye that gives the dissolved agent a pleasant color.

In addition, a fragrance component in dry form is added to the composition.

In a preferred embodiment of the present invention, the enzyme urease is used as the protease: a hydrolytic enzyme from the group of amidases, characterized by the specific property of catalyzing the hydrolytic cleavage of urea into carbon dioxide and ammonia. Any other target enzyme also can be used.

In a preferred embodiment of the invention sodium lauryl sulfate is used as a surfactant. However, it is possible to use any other suitable surfactant.

In the preferred embodiment of the present invention, the “Diamond Blue” dye is used as the dye, which gives the dissolved composition a pleasant blue color. It is possible to use any other suitable dye for these purposes.

In a preferred embodiment of the present invention, the “Ozone” fragrance is used in dry form. In addition, it is possible to use any other suitable fragrances, such as: fragrance “Perfume”, fragrance “Aloe Vera Lemongrass”, fragrance “Marine”, fragrance “Breeze”, fragrance “Lavender”, or other suitable fragrance, as well as fragrances with hypoallergenic properties.

According to the presented technical solution (composition), the enzyme protease (urease) can be used in the concentration range of 2-6 wt. %, preferably 4-5 wt. %, more preferably 5 wt. %.

In the foaming complex, sodium bicarbonate is used in an amount of 33-50 wt. %, preferably 33-45 wt. %, more preferably 33 wt. %; citric acid is taken in an amount of 30-40 wt. %, preferably 25-35 wt. %, more preferably 31.53 wt. %; the surfactant is used in an amount of 15-25 wt. %, preferably 16-20 wt. %, more preferably 18.7 wt. %.

Disodium EDTA (Trilon B) in the proposed compositions is used in an amount of 0.5-1.5 wt. %, more preferably 1.0 wt. %.

Sodium percarbonate as a component with disinfectant properties is included in this composition in the amount of 7-15 wt. %, preferably 8-12 wt. %, more preferably 10 wt. %.

The dye is included in the composition in an amount of 0.03-0.1 wt. %, more preferably 0.07 wt. %.

The fragrance in the means was 0.3-1.0 weight. %, more preferably 0.7 wt. %.

The inventors conducted a series of experiments and tests on the form of presentation of this composition (powder composition with a set of ingredients, liquid, and tablet). Studies have shown the greatest effectiveness of the composition in tablet form. The tablet enters the liquid, dissolves, and releases from the composition a protease which contacts the urine. To ensure that the protease molecules are rapidly distributed throughout the liquid volume and do not accumulate in one place and form separate complexes, the composition comprises a mixture of sodium bicarbonate and citric acid in combination with anionic surfactants (sodium lauryl sulfate). This mixture has a disinfecting and cleansing effect and promotes effective foaming in contact with water. Accordingly, protease (urease) molecules are rapidly distributed throughout the volume and effectively destroy the components of urine. Due to the relatively high concentration of protease and anionic surfactants, the composition has a prolonged effect. In addition, the preparation in the form of tablets contains a definite dosage amount of substances compared, for example, with bulk packaging, where the individual components accumulate and disrupt the content of components in a single dose. This formulation owing to its properties, due to the composition of the components, shape and size of the tablet can be introduced into the lavatory pan both before and after the toilet visit.

To prepare tablets in accordance with the proposed compositions, all its components were thoroughly mixed evenly, formed into tablets by compression, dried and placed in a package.

The following is information confirming carrying out the invention by examples, which, however, does not limit the possible embodiments of the proposed technical solution.

### DETAILED DESCRIPTION

### EXAMPLE 1

To prepare the composition, said components were taken in the given weight ratios. The tablets were then mixed and formed by compression.

| Component | Amount, wt. % |
|---|---|
| Sodium bicarbonate | 30.0 |
| Citric acid | 40.0 |
| Sodium percarbonate | 10.0 |
| Disodium EDTA (Trilon B) | 0.5 |
| Sodium lauryl sulfate | 15.0 |
| Urease (dry) | 4.0 |
| Dye “Diamond Blue” | 0.2 |
| Fragrance “Ozone” (dry) | 0.3 |
| Total amount, % | 100.0 |

### EXAMPLE 2

The components were taken in the weight ratios and formed into tablets.

| Component | Amount, wt. % |
|---|---|
| Sodium bicarbonate | 35.00 |
| Citric acid | 30.00 |
| Sodium percarbonate | 12.00 |
| Disodium EDTA (Trilon B) | 1.50 |
| Sodium lauryl sulfate | 16.50 |
| Urease (dry) | 4.00 |
| Dye “Diamond Blue” | 0.10 |
| Fragrance “Perfume” (dry) | 0.90 |
| Total amount, % | 100.00 |

### EXAMPLE 3

The components were taken in the given weight ratios and formed into tablets.

| Component | Amount, wt. % |
|---|---|
| Sodium bicarbonate | 55.00 |
| Citric acid | 20.00 |
| Sodium percarbonate | 7.00 |
| Disodium EDTA (Trilon B) | 0.50 |
| Sodium lauryl sulfate | 15.00 |
| Urease (dry) | 2.00 |
| Dye “Diamond Blue” | 0.03 |
| Fragrance “Aloe Vera Lemongrass” (dry) | 0.47 |
| Total amount, % | 100.00 |

### EXAMPLE 4

The components were taken in the given weight ratios and formed into tablets.

| Component | Amount, wt. % |
|---|---|
| Sodium bicarbonate | 35.00 |
| Citric acid | 30.00 |
| Sodium percarbonate | 15.00 |
| Disodium EDTA (Trilon B) | 1.40 |
| Urease (dry) | 3.00 |
| Dye “Diamond Blue” | 0.10 |
| Fragrance “Marine” (dry) | 0.50 |
| Total amount, % | 100.00 |

### EXAMPLE 5

The components were taken in the given weight ratios and formed into tablets.

| Component | Amount, wt. % |
|---|---|
| Sodium bicarbonate | 35.0 |
| Citric acid | 20.0 |
| Sodium percarbonate | 11.4 |
| Disodium EDTA (Trilon B) | 1.5 |
| Sodium lauryl sulfate | 25.0 |
| Urease (dry) | 6.0 |
| Dye “Diamond Blue” | 0.1 |
| Fragrance “Breeze” (dry) | 1.0 |
| Total amount, % | 100.0 |

### EXAMPLE 6

The components were taken in the given weight ratios and formed into tablets. This example shows the weight ratios for the best embodiment.

| Component | Amount, wt. % |
|---|---|
| Sodium bicarbonate | 33.00 |
| Citric acid | 31.53 |
| Sodium percarbonate | 10.00 |
| Disodium EDTA (Trilon B) | 1.00 |
| Sodium lauryl sulfate | 18.70 |
| Urease (dry) | 5.00 |
| Dye “Diamond Blue” | 0.07 |
| Fragrance “Lavender” (dry) | 0.70 |
| Total amount, % | 100.00 |

### EXAMPLE 7

The composition in the form of tablets was added to clean water in the lavatory pan before using the toilet. All variants of the composition according to Examples 1-5 showed excellent results: the composition neutralized the odor from the toilet for 6-9 hours. The best, most unexpected effect was provided by the composition according to Example 6: with a single use, the tablet acted for almost 12 hours.

### EXAMPLE 8

A study was conducted to confirm the RESULT of the technical solution. In particular, the total number of microorganisms in 1 m³ of test liquid volume was determined. Sampling was performed on nutrient agar such as meat-and-peptone agar, powder nutrient agar, or fishmeal extract agar. The cultures were incubated at 37° C. for 48±2 h, the number of colonies grown was counted and recalculated per 1 m³ of test fluid volume. Analysis of the test liquid before the application of the disinfectant solution according to the invention showed the presence of an average of 10-12 colonies of microorganisms per Petri dish. After treatment of the test liquid with the solution the absence of growth of pathogenic and opportunistic bacteria was recorded.

### EXAMPLE 9

Flushing from the surface of the lavatory pan was carried out in 1, 3, 6, 9, and 12 hours after application of the tablet form according to the invention using sterile gauze napkins, 5×5 cm in size, soaked in the means according to the invention. The inner surface of the lavatory pan over the border of the convex meniscus of the test liquid was carefully wiped with a gauze napkin. Also flushing from the surface of the recesses of the upper inner part of the lavatory pan was performed in 1, 3, 6, 9, and 12 hours after treatment with the composition according to the invention. After sampling, a gauze napkin was placed in a wide-necked test tube or flask with saline and glass beads and shaken for 10 minutes. The liquid was injected deep into 2 Petri dishes with meat-and-peptone agar (0.5 ml each) and into 2 tubes with 0.5% sugar broth (1 ml each). The cultures were incubated at 37° C. for 48 hours. The lack of growth of pathogenic and opportunistic bacteria was recorded.

Therefore, the use of the disclosed tablets helps to remove odor (as result of presence of urea) from the waterless toilet (or the lavatory pan), due to the decomposition of urea and deodorizing and disinfecting effect of this composition, without having to flush.

1. A tablet to prevent bacterial growth, comprising the enzyme protease, characterized in that it further comprises a complex of sodium bicarbonate, citric acid, and a surfactant for foaming, as well as disodium EDTA (Trilon B), sodium percarbonate, dye and odor and provides neutralization of odors from the toilet by placing in the lavatory pan.

2. The tablet of claim 1, comprising, wt. %:

| | |
|---|---|
| Sodium bicarbonate | 33.0-55.0 |
| acid | |
| Citric | 20.0-40.0 |
| Sodium percarbonate | 7.0-15.0 |
| Disodium EDTA (Trilon B) | 0.5-1.5 |
| Sodium lauryl sulfate | 15.0-25.0 |
| Urease (dry) | 2.0-6.0 |
| Dye | 0.03-0.1 |
| Fragrance (dry) | 0.3-1.0. |

3. The tablet of claim 1, characterized in that it contains as the fragrance an ingredient selected from the group: fragrance “Ozone”, fragrance “Perfume”, fragrance “Aloe Vera Lemongrass”, fragrance “Marine”, fragrance “Breeze”, fragrance “Lavender”, or other suitable fragrance.

4. The tablet of claim 1, characterized in that as the dye contains a dye “Diamond Blue” or any other dye.

5. The tablet of claim 1, where the use of the tablet does not require rinsing with water after using the toilet.

6. A tablet for prevention bacterial growth, comprising components in the following ratio, wt. %:

| | |
|---|---|
| Sodium bicarbonate | 33 |
| Citric acid | 31.53 |
| Sodium percarbonate | 10.0 |
| Disodium EDTA (Trilon B) | 1.00 |
| Sodium lauryl sulfate | 18.70 |
| Urease (dry) | 5.00 |
| Dye | 0.07 |
| Fragrance (dry) | 0.7. |

7. The tablet of claim 6, characterized in that it contains as the fragrance an ingredient selected from the group: fragrance “Ozone”, fragrance “Perfume”, fragrance “Aloe Vera Lemongrass”, fragrance “Marine”, fragrance “Breeze”, fragrance “Lavender”, or other suitable fragrance.

8. The tablet of claim 6, characterized in that that as the dye it contains a dye “Diamond Blue” or any other dye.

9. The tablet of claim 6, characterized in that the use of the tablet does not require rinsing with water after visiting the toilet.
